(19) United States
(12) Patent Application Publication
(10) Pub. No.: US 2025/0069720 A1
(43) Pub. Date: Feb. 27, 2025

(54) INFORMATION PROCESSING DEVICE, INFORMATION PROCESSING METHOD, AND PROGRAM THEREFOR

(71) Applicant: SONY GROUP CORPORATION, TOKYO (JP)

(72) Inventors: MASANORI KATSU, TOKYO (JP); TAKASHI FUJIMOTO, TOKYO (JP)

(21) Appl. No.: 18/724,519

(22) PCT Filed: Dec. 16, 2022
(86) PCT No.: PCT/JP2022/046376

§ 371 Date: Jun. 26, 2024

(30) Foreign Application Priority Data
Jan. 5, 2022 (JP) 2022-000614

(51) Int. Cl. G16H 20/10; G16H 10/20; G16H 15/00; G16H 50/70

(52) U.S. Cl. CPC G16H 20/10; G16H 10/20; G16H 15/00; G16H 50/70

(57) ABSTRACT

An information processing device (20) has a log accumulation unit (21), an abnormality detection unit (23), and an accumulated log reproduction unit (22). The log accumulation unit (21) accumulates vital data of a patient (P) for each medication date as a patient log. The abnormality detection unit (23) detects abnormality data indicating an abnormal value from a patient log. The accumulated log reproduction unit (22) reproduces the patient log so as to have the abnormality data associated with the medication date.

### FIELD

The present disclosure relates to an information processing device, an information processing method, and a program therefor.

### BACKGROUND

A doctor performs medical inquiries, counseling, and the like in order to obtain a diagnosis clue. Patient's physical conditions, subjective symptoms, and the like are grasped through a medical inquiry and the like. Prescription of medicines is also performed on the basis of results of medical inquiries and the like.

### CITATION LIST

### Patent Literature

Patent Literature 1: JP 2015-508281 A

### SUMMARY

### Technical Problem

However, it is difficult for a patient with poor medical knowledge to determine his/her own symptom or change in behavior from an objective viewpoint with the same line of sight as that of a specialist. Depending on a state of a patient's mentality, there occurs a problem of impossibility of describing an accurate state of the patient in words. A change in a disease state may appear in a change in an activity amount or the like in a time zone such as during sleep in which a subjective symptom is difficult to obtain. Therefore, it is very difficult to find an optimum medicine through a medical inquiry, counseling, and the like.

Therefore, the present disclosure proposes an information processing device, an information processing method, and a program therefor that support a diagnosis so that a doctor can perform an accurate diagnosis.

### Solution to Problem

According to the present disclosure, an information processing device is provided that comprises: a log accumulation unit that accumulates vital data of a patient for each medication date as a patient log; an abnormality detection unit that detects abnormality data indicating an abnormal value from the patient log; and an accumulated log reproduction unit that reproduces the patient log so as to have the abnormality data associated with the medication date. According to the present disclosure, an information processing method in which an information process of the information processing device is executed by a computer, and a program causing a computer to perform the information process of the information processing device are provided.

### BRIEF DESCRIPTION OF DRAWINGS

FIG. 1 is a diagram for explaining a background of the present invention.

FIG. 2 is a diagram illustrating an example of a configuration of a diagnosis support system.

FIG. 3 is a diagram illustrating an example of a presentation format of vital data.

FIG. 4 is a diagram illustrating an example in which the vital data is presented as RAW data.

FIG. 5 is a diagram illustrating an example in which the vital data is presented as a relative change from normal time.

FIG. 6 is a diagram illustrating an example in which the vital data is presented as a relative change from the normal time.

FIG. 7 is a diagram illustrating an example of presenting a result of analysis of a tendency of a change in the vital data.

FIG. 8 is a diagram illustrating an example of adding a tag indicating an abnormality content to abnormality data.

FIG. 9 is a diagram illustrating an example of a method of setting a normal cluster.

FIG. 10 is a diagram illustrating an example of data analysis using the normal cluster.

FIG. 11 is a diagram illustrating a presentation example of the abnormality data.

FIG. 12 is a diagram illustrating a presentation example of the abnormality data.

FIG. 13 is a diagram illustrating a presentation example of the abnormality data.

FIG. 14 is a diagram for explaining an example of an assist function for supporting doctor's input.

FIG. 15 is a diagram for explaining another example of the assist function.

FIG. 16 is a diagram illustrating an example of directly designating a range in a data space.

FIG. 17 is a diagram for explaining a question message automatic generation function.

FIG. 18 is a diagram for explaining a relationship between a data range designation method and a template.

FIG. 19 is a diagram for explaining a relationship between the data range designation method and the template.

FIG. 20 is a diagram for explaining a relationship between the data range designation method and the template.

FIG. 21 is a diagram for explaining display of a question history.

FIG. 22 is a diagram for explaining an example of generating observation information using a learning model.

FIG. 23 is a diagram illustrating an example in which a question generated by a doctor and a question based on the observation information are distinguishably displayed.

FIG. 24 is a diagram illustrating an example of a hardware configuration of a server.

### DESCRIPTION OF EMBODIMENTS

In the following, embodiments of the present disclosure will be described in detail with reference to the drawings. In each of the following embodiments, the same parts are denoted by the same reference numerals to omit overlapping description.

Note that the description will be given in the following order.
[1. Background]
[2. Configuration of Diagnosis Support System]
[3. Presentation Format of Vital Data]
   [3-1. Presentation of RAW Data]
   [3-2. Presentation of Relative Change from Normal Time]
   [3-3. Tendency Analysis]
   [3-4. Display of Abnormality Content by Tag]
[4. Detection of Abnormality Data]
   [4-1. Setting of Normal Cluster]
   [4-2. Presentation Example of Abnormality Data]
   [4-3. Doctor's Input Support]
   [4-4. Range Designation in Data Space]
[5. Automatic Generation of Question Message]
[6. Display of Question History]
[7. Generation of Observation Information Using Learning Model]
[8. Example of Hardware Configuration]
[9. Effects]

### 1. Background

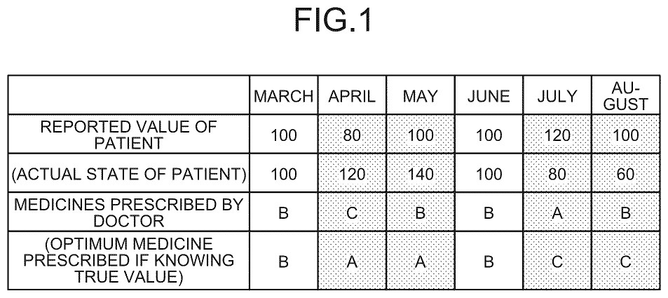

FIG. 1 is a diagram for explaining a background of the present invention.

In a table of FIG. 1, a relationship between a reported value of a patient and a prescribed medicine in each month is described. The table also lists an actual state of a patient (true value) and an optimum medicine prescribed if a doctor knows the true value.

The patient has bipolar disorder. Although the patient repeats manic and depression states, there is no clear regularity in the timing of occurrence of each state. For the sake of explanation, a stable state is described as 100%, the manic state is described as a numerical value larger than 100%, and the depression state is described as a numerical value smaller than 100%. There are three types of medicines A to C as therapeutic agents. The medicine A is a medicine to be prescribed in a manic state. The medicine C is a medicine to be prescribed in a depression state. The medicine B is a medicine to be prescribed in a stable state.

A doctor subscribes a medicine on the basis of patient's reported values and the doctor's own experience. However, it is difficult for a patient with poor medical knowledge to determine his/her own symptom or change in behavior from an objective viewpoint with the same line of sight as that of a specialist. For example, although a patient was in a manic state in April, the patient reported a depression state. Conversely, a patient reported a manic state despite being depressed in July. Due to a failure of appropriate reporting, a medicine having an adverse effect is prescribed, resulting in causing a concern that the symptom is worsened.

In order to return a patient having a mental disease to a normal state, it is necessary to identify an accurate state of a patient and prescribe an appropriate medicine. Depending on a state of a patient's mentality, the patient has a problem of impossibility of describing an accurate state in words, so that objective observation and diagnosis are required. Therefore, in the present disclosure, patient's vital data for each medication date is measured. A true state of a patient is determined on the basis of the measured vital data. By presenting this determination result to a doctor, accurate diagnosis is performed.

In the present disclosure, “vital data” means individual data indicating vital signs such as pulse, blood pressure, respiration, and body temperature. A “vital data group” means a mass of a plurality of pieces of vital data and a plurality of pieces of vital data having similar dispositions.

For example, a plurality of pieces of vital data such as pulse, blood pressure, respiration, and body temperature measured on the same day, a plurality of pieces of vital data having consecutive measurement days, and the like can be referred to as a vital data group in a sense of a mass of a plurality of pieces of vital data. A plurality of pieces of vital data indicating a normal value, a plurality of pieces of vital data indicating an abnormal value, and the like can also be referred to as a vital data group in a sense of a plurality of pieces of vital data having similar dispositions.

In the following, a diagnosis support system of the present disclosure will be described with reference to the drawings.

### 2. Configuration of Diagnosis Support System

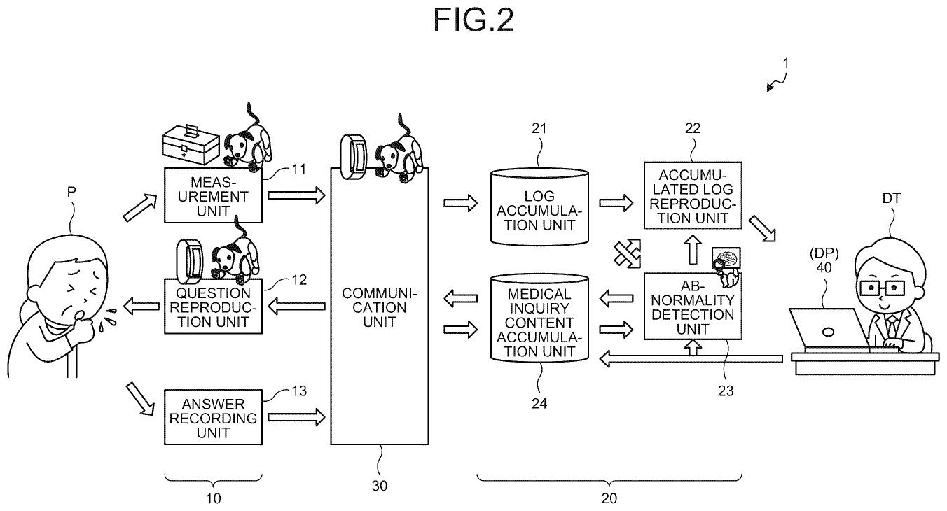

FIG. 2 is a diagram illustrating an example of a configuration of a diagnosis support system 1

The diagnosis support system 1 has a client terminal 10, a server 20, and a medical practitioner terminal 40. The client terminal 10, the server 20, and the medical practitioner terminal 40 are connected via a network. Although in FIG. 2, a communication unit 30 is described as an independent configuration, in practice, communication apparatuses are built in the client terminal 10 and the server 20, respectively. In FIG. 2, communication apparatuses built in the client terminal 10 and the server 20 are collectively described as the communication unit 30.

The client terminal 10 is an information terminal such as a smartphone, a tablet terminal, and a notebook computer. The client terminal 10 is owned by a patient P. The client terminal 10 acquires vital data of the patient P and sequentially transmits the vital data to the server 20. Examples of the vital data to be measured include data such as a heart rate, a heart rate variability, a blood pressure, a deep body temperature, a surface temperature, a body weight, a sleep time, a sleep depth, an activity amount, number of steps consumed calories, a maximum oxygen intake amount, and a blood oxygen concentration.

The client terminal 10 has a measurement unit 11, a question reproduction unit 12, and an answer recording unit 13. The measurement unit 11 has a sensor group that measures vital data of the patient P. The measurement unit 11 tracks a behavior of the patient P and collects vital data. The measurement unit 11 sequentially transmits the measured vital data to the server 20. The question reproduction unit 12 reproduces a question acquired from the server 20 by text, audio, or video. The patient P answers the question using text, voice, button operation, and the like. The answer recording unit 13 records the answer of the patient P. The answer recording unit 13 sequentially transmits the recorded answer to the server 20.

The server 20 is an information processing device that processes various types of information. The server 20 processes and reproduces the vital data acquired from the client terminal 10 into a form that can be determined by a doctor DT. Information presented by the reproduction includes, for example, information such as a behavior of the patient P from a previous visit to a hospital to this visit and a state of the patient P inferred from the behavior of the patient P. The server 20 supports diagnosis of the doctor DT by appropriately processing and presenting the vital data.

The server 20 has a log accumulation unit 21, an accumulated log reproduction unit 22, an abnormality detection unit 23, and a medical inquiry content accumulation unit 24. The log accumulation unit 21 accumulates the vital data of the patient P for each medication date as a patient log. The abnormality detection unit 23 detects vital data (abnormality data) indicating an abnormal value from the patient log. The accumulated log reproduction unit 22 reproduces the patient log so as to have the abnormality data associated with the medication date. The accumulated log reproduction unit 22 transmits the reproduced patient log to the medical practitioner terminal 40.

The medical practitioner terminal 40 is an information terminal such as a smartphone, a tablet terminal, a notebook computer, and a desktop computer. The medical practitioner terminal 40 reproduces the patient log acquired from the accumulated log reproduction unit 22 on a display DP. The doctor DT can ask a question to the patient P on the basis of the patient log presented via the medical practitioner terminal 40. Data of the question is transmitted to the client terminal 10 (the question reproduction unit 12) via the server 20. A patient's answer to the question is transmitted to the medical practitioner terminal 40 via the server 20. The medical inquiry content accumulation unit 24 stores a content of the question and the answer of the patient P to the question in association with each other.

### 3. Presentation Format of Vital Data

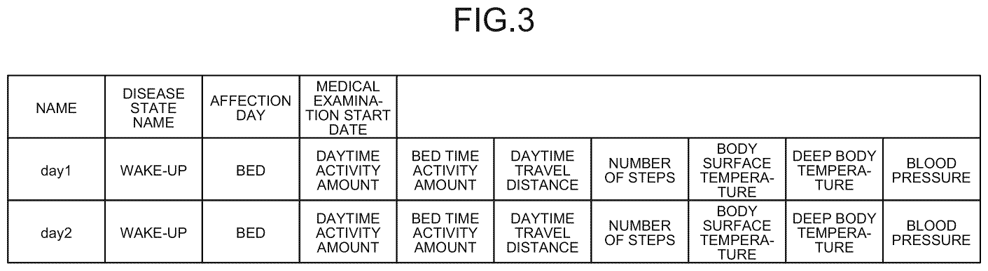

FIG. 3 is a diagram illustrating an example of a presentation format of vital data.

For example, the accumulated log reproduction unit 22 visualizes and presents the vital data in time series. In the example of FIG. 3, vital data is presented as daily data. Possible presentation methods are (i) a method of presenting the vital data as RAW data (data acquired from a sensor group and yet to be processed), (ii) a method of presenting a state of the patient P in a form of a relative change (change amount or change rate) from vital data measured in the normal time, (iii) a method of presenting a result obtained by analyzing a change tendency of the vital data, and the like. Specific examples will be described below.

### 3-1. Presentation of RAW Data

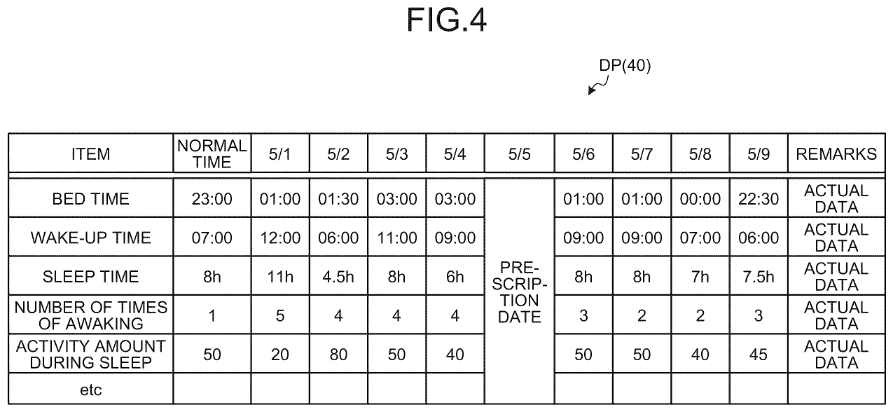

FIG. 4 is a diagram illustrating an example in which the vital data is presented as RAW data.

In the example of FIG. 4, the vital data is presented in a table form. At a left end of the table, items of vital data are listed. Daily vital data is displayed from a left side to a right side of the table. In the table, a prescription date of a medicine is specified. In the table, vital data of the patient P in the normal time is also displayed as reference data.

Although in the example of FIG. 4, the daily vital data is presented, the presentation format is not limited thereto. In some diseases, symptoms appear slowly on a weekly basis or a monthly basis. In that case, an average value of weekly or monthly vital data may be presented. A management user (e.g., the doctor DT) of the medical practitioner terminal 40 may be able to select how to summarize the data. The vital data of a part having no abnormality may be compiled and displayed.

3-2. Presentation of Relative Change from Normal Time

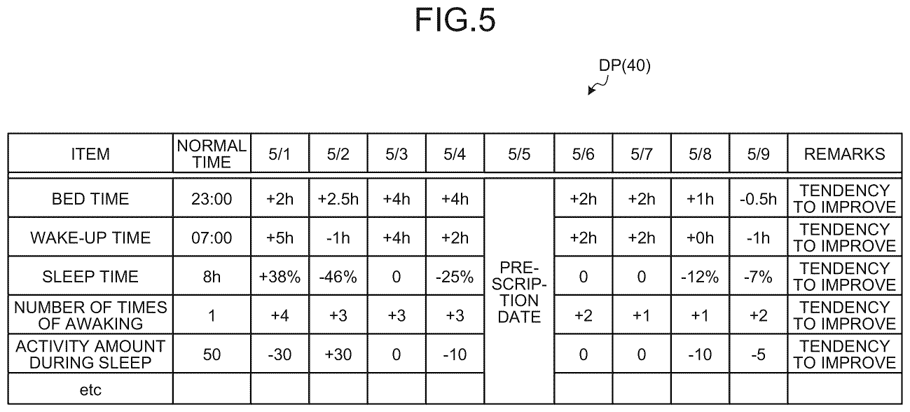

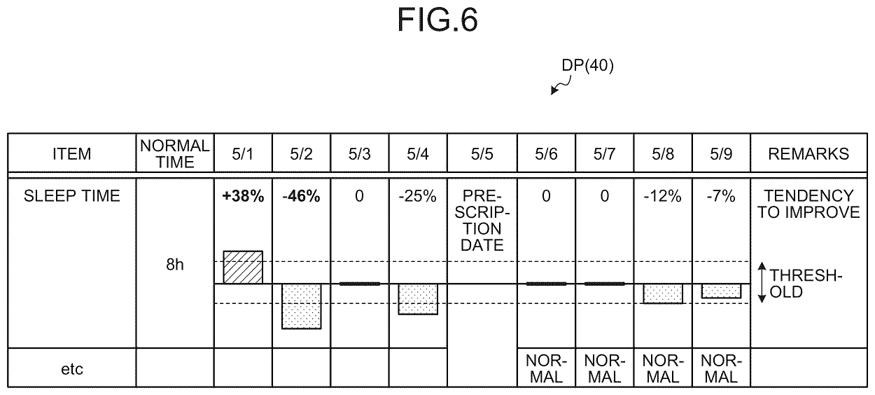

FIGS. 5 and 6 are diagrams illustrating examples in which vital data is presented as a relative change from the normal time.

In the example of FIG. 5, regarding bed time,

wake-up time, the number of times of awakening, and an activity amount during sleep, a change amount of the vital data from the normal time is presented. For the sleep time, a change rate of the vital data from the normal time is presented. Although in the example of FIG. 5, a daily change amount or change rate of the vital data is presented, a weekly or monthly change amount or change rate of the vital data may be presented.

In the example of FIG. 6, the vital data is presented in a form of a bar graph. In the bar graph, a size of the vital data is also shown as a relative change from the normal time. While in the example of FIG. 6, sleep time is shown as one item of the vital data, vital data of other items is also presented in a similar manner. With respect to the sleep time, while a change rate from the normal time is presented, whether to present a change rate or a change amount may be different for each item.

The accumulated log reproduction unit 22 can present abnormality data in a mode that enables distinction from normal data (vital data indicating a normal value). In the example of FIG. 6, a range of the normal value (a normal range) is clearly indicated by a dotted line or the like. With respect to the abnormality data, color of the bar graph and color of the numerical value of the vital data can be made different from those of the normal data. The normal data may be also clearly indicated to be normal by tagging or color-coding.

### 3-3. Tendency Analysis

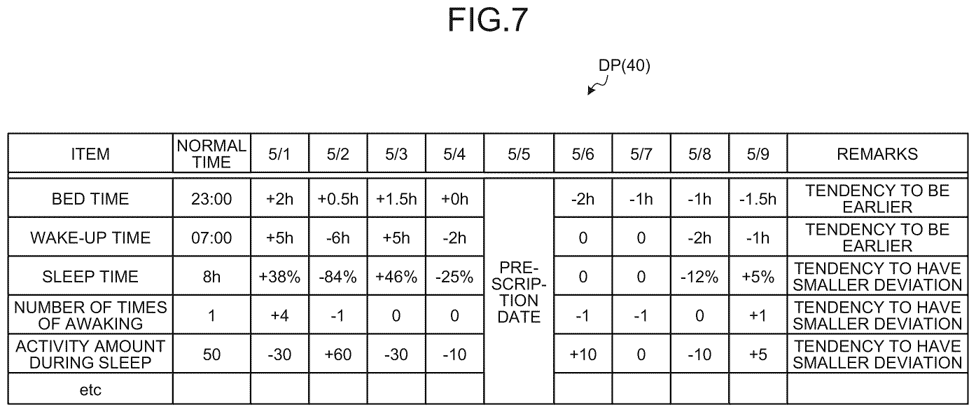

FIG. 7 is a diagram illustrating an example of presenting a result obtained by analyzing a tendency of a change in the vital data.

In the example of FIG. 7, the vital data is presented as a relative change from a previous measurement day. In a remarks column, the tendency of the change in the vital data obtained from a series of measurement results is described. In the example of FIG. 7, the tendency of the change is analyzed on the basis of a daily increase/decrease of the vital data. However, in some diseases, symptoms slowly appear on a weekly basis or a monthly basis. In that case, the tendency of the change may be analyzed on the basis of a weekly or monthly average amount of change or change rate of the vital data.

### 3-4. Display of Abnormality Content by Tag

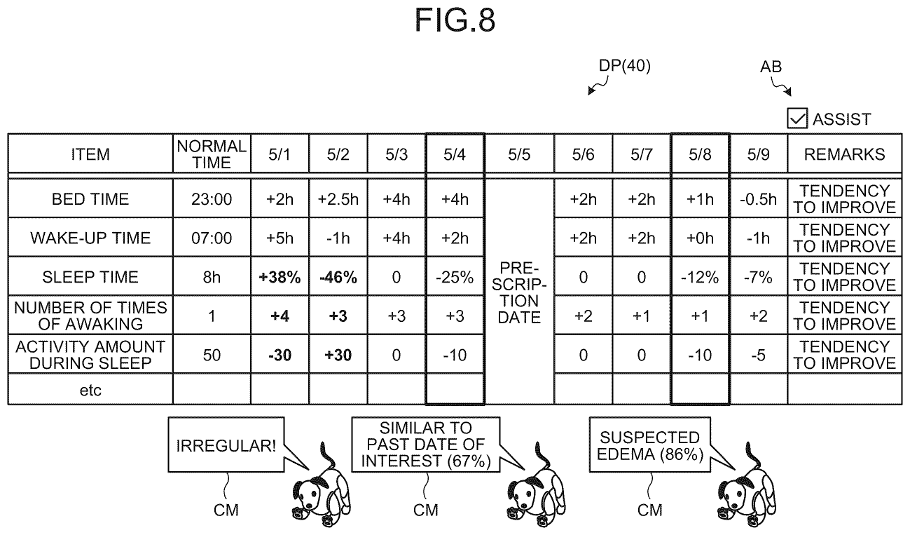

FIG. 8 is a diagram illustrating an example of adding a tag CM indicating an abnormality content to abnormality data.

In the table of FIG. 8, an assist button AB is provided. When the assist button AB is turned on, a vital data group (abnormality data group) indicating an abnormal value is highlight-displayed. Examples of a highlight-displaying method include a method of varying color, size, and thickness of characters, and a method of surrounding the vital data group with a frame. The accumulated log reproduction unit 22 presents the abnormality data in a mode that enables distinction from the normal data by highlight-display.

The abnormality data group to be highlight-displayed includes (A) vital data statistically estimated to be abnormal, (B) vital data similar to data of interest in the past, and (C) vital data similar to vital data associated with a subjective symptom (such as edema). The data of interest means vital data to which the doctor DT has paid attention for some reason or another. It does not matter whether or not a cause of the abnormality is identified for the data of interest.

For example, the abnormality data group (A) is extracted using a statistical method such as a local outlier factor (LOF) method. The abnormality data group of (B) is extracted on the basis of a learning result of a past history of interest of the doctor DT. The abnormality data group of (C) is extracted on the basis of a learning result of a history of ascertainment between the doctor DT and the patient P.

The accumulated log reproduction unit 22 can present the tag CM indicating a content of abnormality in association with the abnormality data. With respect to the abnormality data groups of (B) and (C), a degree of similarity can be displayed together with a content of the abnormality. A known method can be used to calculate the degree of similarity. In the example of FIG. 8, it is described that with respect to the vital data group of May 4, the degree of similarity to a past data of interest group is 67%. In addition, it is described that a possibility of occurrence of the symptom of edema is 86% with respect to the vital data group of May 8.

### 4. Detection of Abnormality Data

### 4-1. Setting of Normal Cluster

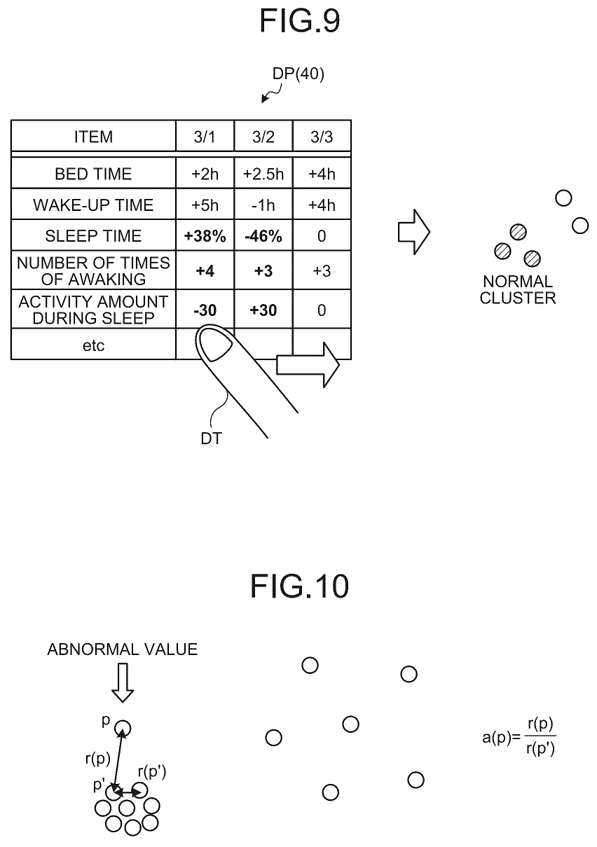

FIG. 9 is a diagram illustrating an example of a method of setting a normal cluster.

The abnormality data is identified as an outlier from a normal data group. The normal data group means a normal vital data group indicating a normal value. The accumulated log reproduction unit 22 presents a UI for identifying a normal data group as a normal cluster from the vital data group of the patient P. For example, the doctor DT designates a normal data group by touch operation on a reproduction screen of the patient log. The accumulated log reproduction unit 22 detects the normal data group designated by the doctor DT as a normal cluster.

In a common abnormality detection technique, an outlier in a distribution is detected as an abnormal value. In the present disclosure, the doctor DT manually designates a normal data group on the basis of medical knowledge without using such a common abnormality detection technique. This is because if there is no input of a stable section by the doctor DT, for a patient whose bad state becomes common, vital data of the stable state that is occasionally observed is recognized as abnormality data.

FIG. 10 is a diagram illustrating an example of data analysis using the normal cluster.

Detection of an abnormal value is performed using a statistical method such as the LOF method. Whether or not a value is an abnormal value is determined on the basis of a distance from the normal cluster in a data space. The abnormality detection unit 23 detects, as abnormality data, vital data whose distance from the normal cluster exceeds an allowable criterion.

For example, with respect to vital data (target data p) as a target for abnormality determination, the abnormality detection unit 23 detects vital data (first data p′) closest to the target data p in the normal cluster and vital data (second data) closest to the first data p′ in the normal cluster. The abnormality detection unit 23 calculates a distance r(p) between the target data p and the first data p′ and a distance r(p′) between the first data p′ and the second data. The abnormality detection unit 23 calculates an abnormality degree a(p) on the basis of a (p)=r(p)/r(p′).

The abnormality detection unit 23 detects, as abnormality data, vital data whose abnormality degree a(p) exceeds an allowable criterion (e.g., threshold). The allowable criterion is appropriately set according to a type of disease, characteristics of the patient P (age, sex, temperament, physical strength, weight of symptom, etc.), and the like.

### 4-2. Presentation Example of Abnormality Data

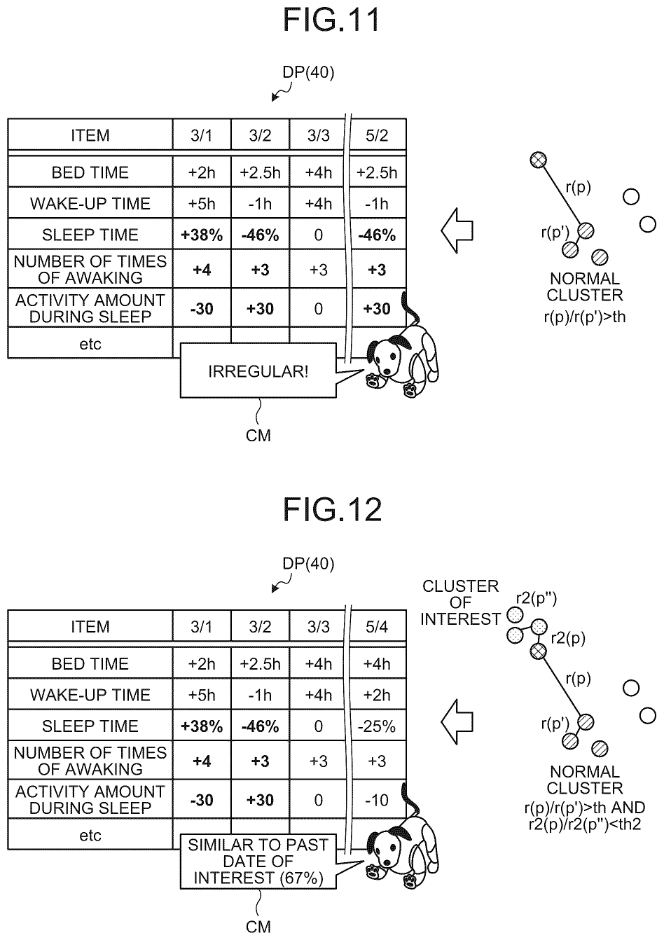

FIG. 11 to FIG. 13 are diagrams illustrating presentation examples of the abnormality data.

In the example of FIG. 11, a vital data group (abnormality data group) whose distance from the normal cluster exceeds a threshold th is highlight-displayed. The tag CM indicating a content of abnormality is added to the abnormality data group.

In the example of FIG. 12, a vital data group similar to past data of interest is highlight-displayed. The tag CM describes that the highlight-displayed vital data group is similar to the past data of interest, and a degree of similarity to the data of interest. Whether or not the data is similar to the data of interest is determined on the basis of a distance from a cluster of interest in the data space. The cluster of interest means a vital data group (data of interest group) to which the doctor DT has paid attention in the past.

For example, the abnormality detection unit 23 detects vital data (third data p″) closest to the target data p in the cluster of interest and vital data (fourth data) closest to the third data p″ in the cluster of interest. The abnormality detection unit 23 calculates a distance r2(p) between the target data p and the third data p″ and a distance r2(p″) between the third data p″ and the fourth data. The abnormality detection unit 23 calculates a degree of interest b(p) on the basis of b(p)=r2(p)/r2(p″). The abnormality detection unit 23 detects target data p having the degree of interest b(p) exceeding a predetermined criterion (e.g., a threshold th2) as vital data similar to the data of interest.

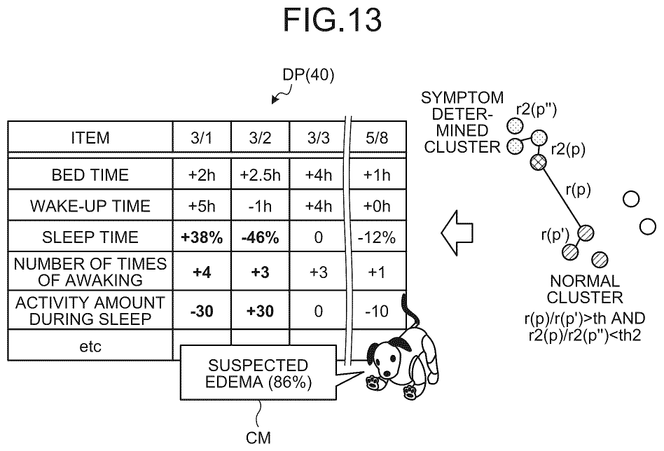

In the example of FIG. 13, a vital data group similar to vital data (symptom determined data) associated with subjective symptoms is highlight-displayed. In the tag CM, subjective symptoms associated with the similar symptom determined data and a degree of similarity to the symptom determined data are described. Whether the data is similar to the symptom determined data or not is determined on the basis of a distance from a symptom determined cluster in the data space. The symptom determined cluster means a vital data group (symptom determined data group) associated with subjective symptoms. A determination method therefor is similar to that of the case of the data of interest described above.

### 4-3. Doctor's Input Support

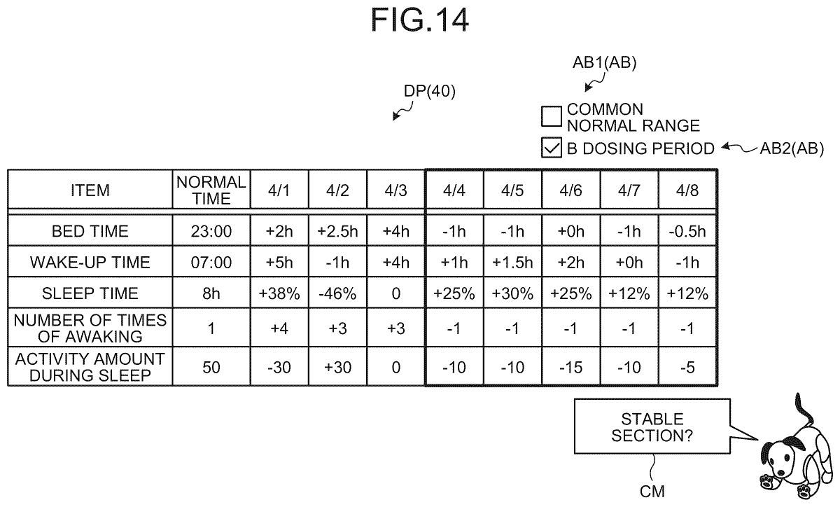

FIG. 14 is a diagram for explaining an example of an assist function for supporting input of the doctor DT.

The accumulated log reproduction unit 22 presents the assist button AB on the UI. FIG. 14 illustrates a first assist button AB1 and a second assist button AB2 as an example of the assist button AB. The first assist button AB1 has a function of extracting a vital data group included in a common normal range from the vital data group of the patient P. The second assist button AB2 has a function of extracting a vital data group of a specific dosing period from the vital data group of the patient P.

The common normal range means a range of vital data that is generally recognized as a normal value. The common normal range is defined on the basis of vital data of healthy persons. The doctor DT can customize the common normal range. The doctor DT can select a type of medicine displayed on the second assist button AB by pull-down.

When the doctor DT presses the first assist button AB1, the accumulated log reproduction unit 22 presents a vital data group similar to the vital data of healthy persons as a stable candidate. The doctor DT determines whether to approve the presented vital data group as a normal cluster or not. The abnormality detection unit 23 identifies the stable candidate approved by the doctor DT as a normal cluster.

When the doctor DT presses the second assist button AB2, the accumulated log reproduction unit 22 presents, as a stable candidate, a vital data group with a medication period of a medicine designated by the doctor DT. The doctor DT determines whether to approve the presented vital data group as a normal cluster or not. The abnormality detection unit 23 identifies the stable candidate approved by the doctor DT as a normal cluster.

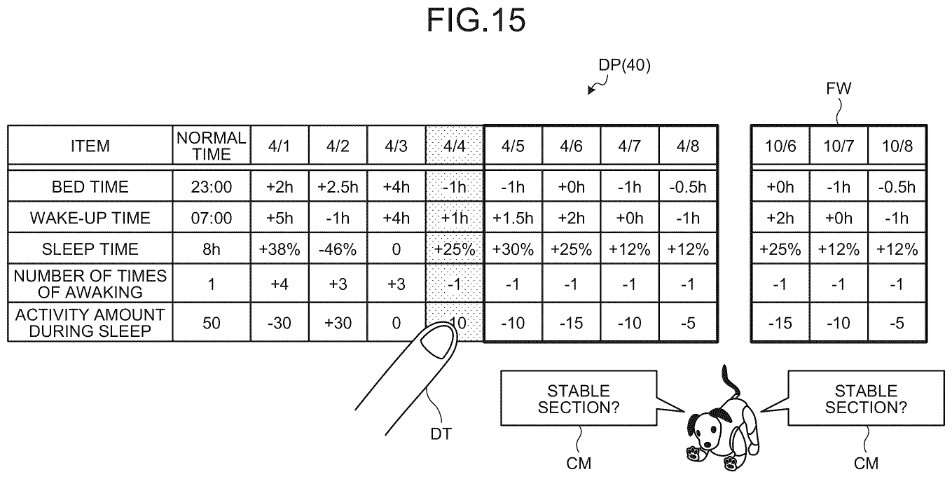

FIG. 15 is a diagram for explaining another example of the assist function.

In the example of FIG. 15, a vital data group in a range similar to a range directly designated by the doctor DT is also identified as a normal cluster. For example, the accumulated log reproduction unit 22 presents a vital data group similar to a vital data group designated by the doctor DT as a stable candidate. The doctor DT determines whether to approve the presented vital data group as a normal cluster or not. The abnormality detection unit 23 identifies the stable candidate approved by the doctor DT as a normal cluster together with the vital data group designated by the doctor DT.

The vital data group extracted by the assist button AB may include a plurality of vital data groups whose medication dates are not consecutive. The vital data groups with medication dates apart from each other can be displayed in a floating window FW or the like.

### 4-4. Range Designation in Data Space

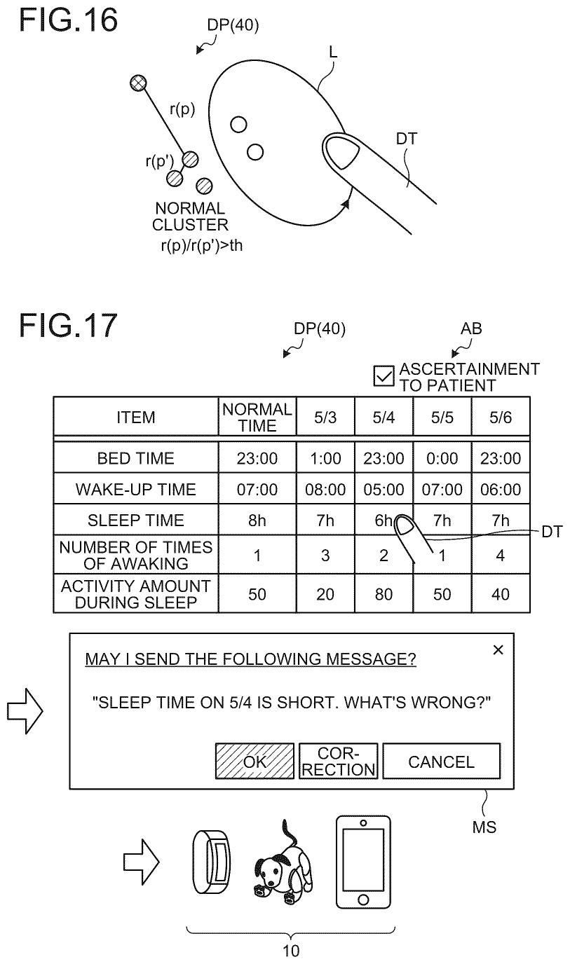

FIG. 16 is a diagram illustrating an example of directly designating a range in the data space.

The accumulated log reproduction unit 22 can display the vital data group of the patient P in the data space. The doctor DT can designate a range of the normal cluster directly in the data space. For example, the accumulated log reproduction unit 22 presents a data space of vital data on the UI. The doctor DT identifies a range indicating a normal value using a lasso L or the like. The abnormality detection unit 23 identifies a vital data group included in the space designated by the doctor DT as a normal cluster.

### 5. Automatic Generation of Question Message

FIG. 17 is a diagram for explaining a question message automatic generation function.

The medical inquiry content accumulation unit 24 can generate a question message MS regarding the data range designated by the doctor DT. When the doctor DT selects specific vital data on the display DP, the medical inquiry content accumulation unit 24 questions the patient P using a template prepared in advance.

In the example of FIG. 17, the doctor DT taps data of the sleep time of May 4. The medical inquiry content accumulation unit 24 generates a message MS “Sleep time on (May 4) is (short). What's wrong?” using a template for a question about the sleep time. The words in parentheses in the message MS are input by the doctor DT.

The doctor DT determines whether to approve the generated message MS or not. The medical inquiry content accumulation unit 24 selectively presents the message MS approved by the doctor DT to the patient P. The message MS transmitted to the patient P is reproduced by the question reproduction unit 12. When receiving correction operation from the doctor DT, the medical inquiry content accumulation unit 24 corrects the message MS according to the correction operation.

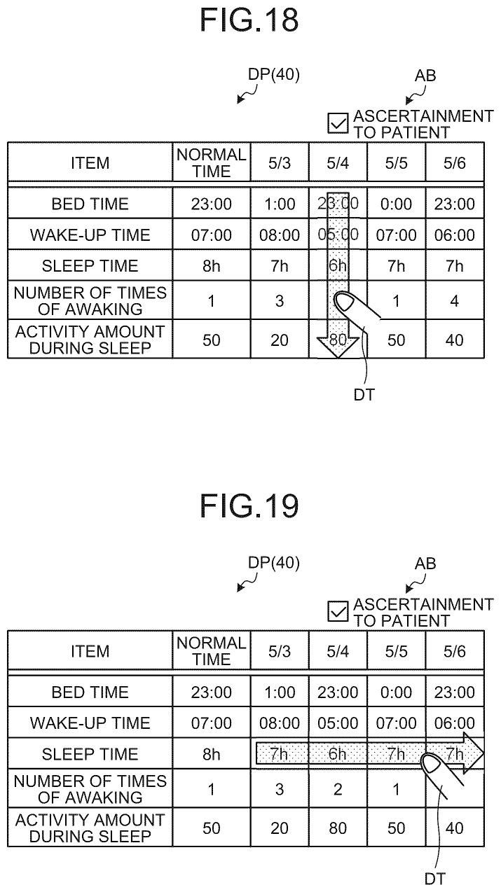

The medical inquiry content accumulation unit 24 can change the template of the message MS on the basis of a data range designation method by the doctor DT. FIG. 18 to FIG. 20 are diagrams for explaining a relationship between the data range designation method and the template.

In the example of FIG. 18, the doctor DT slides a finger in a vertical direction. In this case, the medical inquiry content accumulation unit 24 determines that the doctor DT is going to ask a question not about an individual data item but about a specific date in a vague manner. The medical inquiry content accumulation unit 24 generates a message MS “What happened on (May 4)?” by using a template for vaguely asking a question about a daily behavior.

In the example of FIG. 19, the doctor DT slides the finger in a lateral direction. In this case, the medical inquiry content accumulation unit 24 determines that the doctor DT intends to ask a question about a specific data item extending over a plurality of days. The medical inquiry content accumulation unit 24 generates a message MS “(Recent) sleep time is (short), what's wrong?” using a template for asking a question about a specific data item over a plurality of days.

The words in parentheses in the message MS are input by the doctor DT. For example, while a specific period, such as “recent” in a case where several days leading to the present are designated, or “X/X to Y/Y” in a case where the past several days are designated, is input as an initial value to the template, a drop-down menu or an arbitrary term may be input instead.

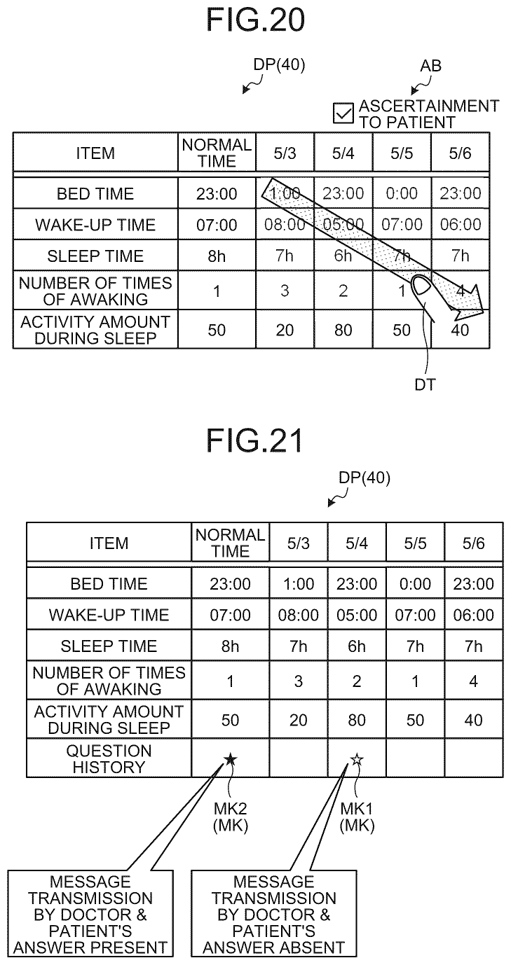

In the example of FIG. 20, the doctor DT slides the finger obliquely. In this case, the medical inquiry content accumulation unit 24 determines that the doctor intends to ask a question about a plurality of data items extending over a plurality of days. The medical inquiry content accumulation unit 24 generates a message MS “How is going (lately)?” using a template for asking a question about a plurality of data items over a plurality of days. The words in parentheses in the message MS are input by the doctor DT. While a specific period, such as “recent” in a case where several days leading to the present are selected, or “X/X to Y/Y” in a case where the past several days are selected, is input as the initial value to the template, the drop-down menu or an arbitrary term may be input instead.

### 6. Display of Question History

FIG. 21 is a diagram for explaining display of a question history.

The accumulated log reproduction unit 22 can display a history of a question to the patient P on the UI together with the patient log. For example, a mark MK is attached to a part where a question to the patient P has been sent. A display mode (color, shape, etc.) of the mark MK may differ depending on presence or absence of an answer from the patient P. In the example of FIG. 21, a white mark MK1 indicates a case where there is no answer from the patient P. A black mark MK2 indicates a case where there is an answer from the patient P.

### 7. Generation of Observation Information Using Learning Model

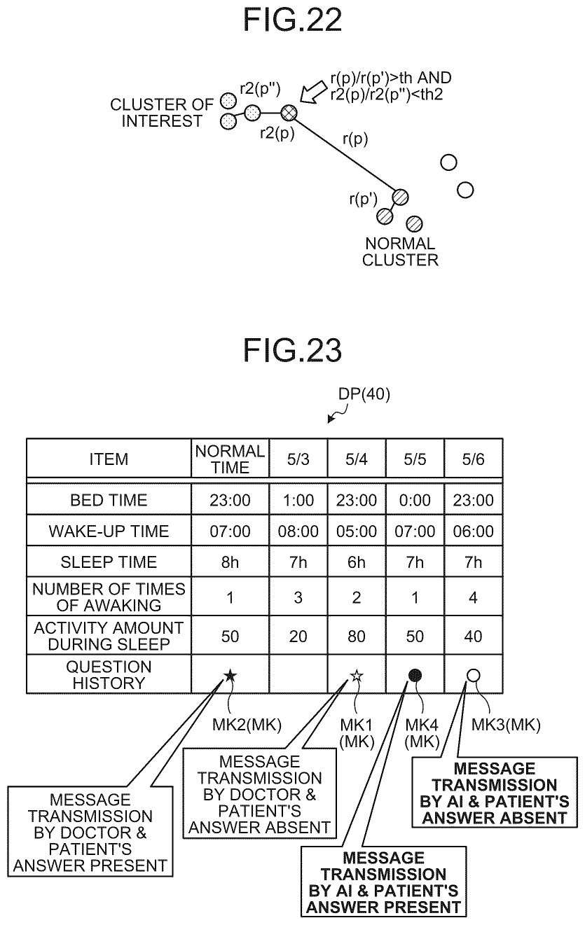

FIG. 22 is a diagram for explaining an example of generating observation information using a learning model.

The abnormality detection unit 23 can generate the observation information according to the patient log using a neural network (observation model) that has learned observation of the doctor DT. The observation information means information indicating an opinion from a medical viewpoint. The learning can be performed using the data accumulated in a medical inquiry content accumulation unit 4 as a learning data set.

In the example of FIG. 22, the abnormality detection unit 23 learns an observation of the doctor DT with respect to the data of interest. When detecting vital data similar to the data of interest from the vital data group of the patient P, the abnormality detection unit 23 generates observation information that the vital data is similar to the data of interest. The medical inquiry content accumulation unit 24 generates a question message MS on the basis of the generated observation information and transmits the message to the client terminal 10.

For example, it is assumed that the data of interest is vital data with a short sleep time, and that it is found from a learning result that the doctor DT pays attention to the sleep time. In this case, the medical inquiry content accumulation unit 24 generates a message MS “(Sleep time) of (yesterday) is (short). What's wrong?” according to the past message history of the doctor DT.

In the above example, the question is presented to the patient P without approval of the doctor DT. However, the doctor DT may select a question to be presented to the patient P. For example, the abnormality detection unit 23 presents the generated observation information to the doctor DT. The doctor DT determines whether to approve the presented observation information or not. The medical inquiry content accumulation unit 24 generates a message MS to the patient P on the basis of the observation information approved by the doctor DT. As a result, the abnormality detection unit 23 can selectively present a question based on the observation information approved by the doctor DT to the patient P.

The abnormality detection unit 23 can generate answer information according to the patient log by using a neural network (answer model) that has learned the answer of the patient P to the question. The learning can be performed using the data accumulated in the medical inquiry content accumulation unit 4 as a learning data set.

For example, it is assumed that it is found from a learning result that the patient has answered the symptom of edema to a question about the vital data selected by the doctor DT. In this case, the abnormality detection unit 23 generates answer information of “suspected edema” according to a past answer history of the patient P. The answer information automatically generated by the abnormality detection unit 23 is transmitted to the medical practitioner terminal 40 together with the answer generated by the patient P.

The accumulated log reproduction unit 22 can present a history of a question in association with a patient log in a mode in which a question generated by the doctor DT and a question based on observation information are distinguishable. FIG. 23 is a diagram illustrating an example in which a question generated by the doctor DT and a question based on the observation information are distinguishably displayed.

In the example of FIG. 23, a star-shaped mark MK is attached to the question generated by the doctor DT. A circular mark MK is attached to a question automatically generated on the basis of observation information generated by the abnormality detection unit 23. A display mode (color, shape, etc.) of the mark MK differs depending on presence or absence of an answer from the patient P. For example, white marks MK1 and MK3 indicate a case where there is no answer from the patient P. Black marks MK2 and MK4 indicate a case where there is an answer from the patient P.

### 8. Example of Hardware Configuration

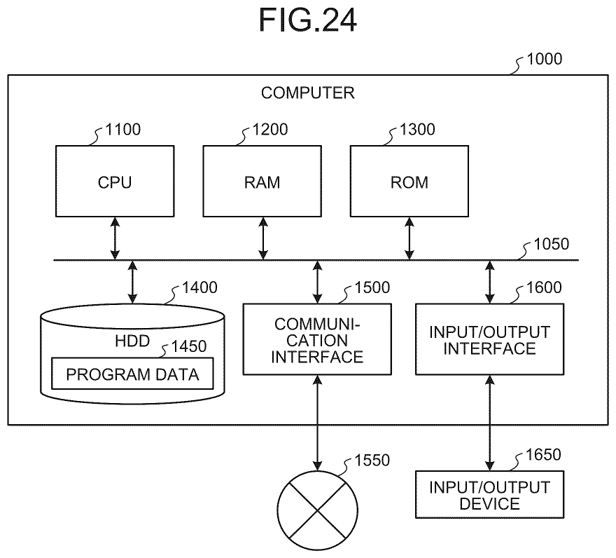

FIG. 24 is a diagram illustrating an example of a hardware configuration of the server 20.

Information processing of the server 20 is realized by, for example, a computer 1000. The computer 1000 includes a central processing unit (CPU) 1100, a random access memory (RAM) 1200, a read only memory (ROM) 1300, a hard disk drive (HDD) 1400, a communication interface 1500, and an input/output interface 1600. Each unit of the computer 1000 is connected by a bus 1050.

The CPU 1100 operates on the basis of a program (program data 1450) stored in the ROM 1300 or the HDD 1400, and controls each unit. For example, the CPU 1100 develops the program stored in the ROM 1300 or the HDD 1400 into the RAM 1200, and executes processing corresponding to various programs.

The ROM 1300 stores a boot program such as a basic input output system (BIOS) executed by the CPU 1100 when the computer 1000 is activated, a program depending on hardware of the computer 1000, and the like.

The HDD 1400 is a non-transitory computer-readable recording medium that non-transiently records a program executed by the CPU 1100, data for use by the program, and the like. Specifically, the HDD 1400 is a recording medium that records an information processing program according to the present embodiment as an example of the program data 1450.

The communication interface 1500 is an interface for the computer 1000 to connect to an external network 1550 (e.g., the Internet). For example, the CPU 1100 receives data from other apparatus or transmits data generated by the CPU 1100 to other apparatus via the communication interface 1500.

The input/output interface 1600 is an interface for connecting an input/output device 1650 and the computer 1000. For example, the CPU 1100 receives data from an input device such as a keyboard or a mouse via the input/output interface 1600. In addition, the CPU 1100 transmits data to an output device such as a display device, a speaker, or a printer via the input/output interface 1600. Furthermore, the input/output interface 1600 may function as a media interface that reads a program or the like recorded in a predetermined recording medium (medium). The medium is, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like.

For example, in a case where the computer 1000 functions as the server 20 according to the embodiment, the CPU 1100 of the computer 1000 implements the functions of the above-described units by executing the information processing program loaded on the RAM 1200. In addition, the HDD 1400 stores the information processing program, various models, and various data according to the present disclosure. Note that although the CPU 1100 reads the program data 1450 from the HDD 1400 and executes the program data, as another example, programs may be acquired from other device via the external network 1550.

### 9. Effects

The server 20 includes the log accumulation unit 21, the abnormality detection unit 23, and the accumulated log reproduction unit 22. The log accumulation unit 21 accumulates the vital data of the patient P for each medication date as a patient log. The abnormality detection unit 23 detects abnormality data indicating an abnormal value from the patient log. The accumulated log reproduction unit 22 reproduces the patient log so as to have the abnormality data associated with the medication date. In the information processing method of the present disclosure, the processing of the server 20 is executed by the computer 1000. The program of the present disclosure causes the computer 1000 to implement the processing of the server 20.

According to this configuration, a relationship between a medicine taken and a change in a symptom of the patient P is objectively grasped. Therefore, an accurate diagnosis can be performed.

The abnormality detection unit 23 generates observation information according to the patient log using an observation model that has learned observation of the doctor DT.

According to this configuration, the change in the symptoms appearing in the patient log is appropriately reflected in the observation information. Since the observation information is automatically generated on the basis of a monitoring result of the patient log, a burden on the doctor DT monitoring the patient log is reduced. In addition, the change in the symptom of the patient P is less likely to be overlooked.

The abnormality detection unit 23 selectively presents a question based on observation information approved by the doctor DT to the patient P.

According to this configuration, an inappropriate question is suppressed from reaching the patient P.

The abnormality detection unit 23 generates answer information according to the patient log by using an answer model that has learned an answer of the patient P to the question.

According to this configuration, it is possible to suppress an inappropriate diagnosis due to waver in determination of the patient P. Since the answer information is automatically generated, a burden on the patient P who answers is reduced.

The accumulated log reproduction unit 22 presents a history of a question in association with a patient log in a mode in which a question generated by the doctor DT and a question based on observation information are distinguishable.

According to this configuration, the doctor DT can appropriately determine priority of a question.

The server 20 includes the medical inquiry content accumulation unit 24. The medical inquiry content accumulation unit 24 stores a content of the question and the answer of the patient P to the question in association with each other.

According to this configuration, the medical inquiry content accumulation unit 24 compiles data of a question and an answer. If the data compiled in the medical inquiry content accumulation unit 24 is used for machine learning, an appropriate answer model is generated.

The medical inquiry content accumulation unit 24 generates a question message MS regarding a data range designated by the doctor DT.

According to this configuration, the doctor DT can question the patient P only by designating a data range of interest without creating the message MS by himself/herself.

The medical inquiry content accumulation unit 24 changes the template of the message MS on the basis of the data range designation method.

According to this configuration, an appropriate template is selected without using complicated machine learning.

The accumulated log reproduction unit 22 presents the abnormality data in a mode that enables distinction from the normal data.

According to this configuration, a relationship between a medication date and abnormality data is easily grasped.

The accumulated log reproduction unit 22 presents the tag CM indicating a content of abnormality in association with the abnormality data.

According to this configuration, it is possible to accurately grasp what kind of abnormality has occurred.

The accumulated log reproduction unit 22 presents a UI for identifying a normal vital data group as a normal cluster from the vital data group of the patient P.

According to this configuration, it is possible to identify an appropriate normal cluster for each patient P.

The accumulated log reproduction unit 22 presents a vital data group similar to vital data of healthy persons as a stable candidate. The abnormality detection unit 23 identifies the stable candidate approved by the doctor DT as a normal cluster.

According to this configuration, it is possible to identify a normal cluster with ease.

The accumulated log reproduction unit 22 presents, as a stable candidate, a vital data group with a medication period of a medicine designated by the doctor DT. The abnormality detection unit 23 identifies the stable candidate approved by the doctor DT as a normal cluster.

According to this configuration, it is possible to identify a normal cluster with ease.

The accumulated log reproduction unit 22 presents a vital data group similar to the vital data group designated by the doctor DT as a stable candidate. The abnormality detection unit 23 identifies the stable candidate approved by the doctor DT as a normal cluster together with the vital data group designated by the doctor DT.

According to this configuration, it is possible to identify a normal cluster with ease.

The accumulated log reproduction unit 22 presents a data space of vital data. The abnormality detection unit 23 identifies a vital data group included in the space designated by the doctor DT as a normal cluster.

According to this configuration, it is possible to identify a normal cluster with ease.

Note that the effects described in the present specification are examples only and are not limited, and other effects may be provided.

### Supplementary Note

Note that the present technique can also have the following configurations.
(1)

An information processing device comprising:
a log accumulation unit that accumulates vital data of a patient for each medication date as a patient log;
an abnormality detection unit that detects abnormality data indicating an abnormal value from the patient log; and
an accumulated log reproduction unit that reproduces the patient log so as to have the abnormality data associated with the medication date.
(2)

The information processing device according to (1), wherein
the abnormality detection unit generates observation information according to the patient log using an observation model that has learned observation of a doctor.
(3)

The information processing device according to (2), wherein
the abnormality detection unit selectively presents a question based on the observation information approved by the doctor to the patient.
(4)

The information processing device according to (2) or (3), wherein
the abnormality detection unit generates answer information according to the patient log by using an answer model that has learned an answer of the patient to a question.
(5)

The information processing device according to (4), wherein
the accumulated log reproduction unit presents, in a mode in which a question generated by the doctor and a question based on the observation information are distinguishable, a history of the question in association with the patient log.
(6)

The information processing device according to (4) or (5), comprising:
a medical inquiry content accumulation unit that stores a content of the question and an answer of the patient to the question in association with each other.
(7)

The information processing device according to (6), wherein
the medical inquiry content accumulation unit generates a question message regarding a data range designated by the doctor.
(8)

The information processing device according to (7), wherein
the medical inquiry content accumulation unit changes a template of the message on the basis of a designation method of the data range.
(9)

The information processing device according to any one of (1) to (8), wherein
the accumulated log reproduction unit presents the abnormality data in a mode that enables distinction from normal data.
(10)

The information processing device according to any one of (1) to (9), wherein
the accumulated log reproduction unit presents a tag indicating a content of abnormality in association with the
(11)

The information processing device according to any one of (1) to (10), wherein

the accumulated log reproduction unit presents a UI for identifying a normal vital data group from a vital data group of the patient as a normal cluster.
(12)

The information processing device according to (11), wherein
the accumulated log reproduction unit presents a vital data group similar to vital data of a healthy person as a stable candidate, and
the abnormality detection unit identifies the stable candidate approved by a doctor as the normal cluster.
(13)

The information processing device according to (11), wherein
the accumulated log reproduction unit presents, as a stable candidate, a vital data group with a medication period of a medicine designated by a doctor, and
the abnormality detection unit identifies the stable candidate approved by the doctor as the normal cluster.
(14)

The information processing device according to (11), wherein
the accumulated log reproduction unit presents, as a stable candidate, a vital data group similar to a vital data group designated by a doctor, and
the abnormality detection unit identifies the stable candidate approved by the doctor as the normal cluster together with the vital data group designated by the doctor.
(15)

The information processing device according to (11), wherein
the accumulated log reproduction unit presents a data space of the vital data, and
the abnormality detection unit identifies a vital data group included in a space designated by a doctor as the normal cluster.
(16)

An information processing method executed by a computer, the method comprising:
accumulating vital data of a patient for each medication date as a patient log;
detecting abnormality data indicating an abnormal value from the patient log; and
reproducing the patient log so as to have the abnormality data associated with the medication date.
(17)

A program for causing a computer to implement:
accumulating vital data of a patient for each medication date as a patient log;
detecting abnormality data indicating an abnormal value from the patient log; and
reproducing the patient log so as to have the abnormality data associated with the medication date.

### REFERENCE SIGNS LIST

20 SERVER (INFORMATION PROCESSING DEVICE)
21 LOG ACCUMULATION UNIT
22 ACCUMULATED LOG REPRODUCTION UNIT
23 ABNORMALITY DETECTION UNIT
24 MEDICAL INQUIRY CONTENT ACCUMULATION UNIT
DT DOCTOR
MS MESSAGE
P PATIENT

1. An information processing device comprising:
a log accumulation unit that accumulates vital data of a patient for each medication date as a patient log;
an abnormality detection unit that detects abnormality data indicating an abnormal value from the patient log; and
an accumulated log reproduction unit that reproduces the patient log so as to have the abnormality data associated with the medication date.

2. The information processing device according to claim 1, wherein
the abnormality detection unit generates observation information according to the patient log using an observation model that has learned observation of a doctor.

3. The information processing device according to claim 2, wherein
the abnormality detection unit selectively presents a question based on the observation information approved by the doctor to the patient.

4. The information processing device according to claim 2, wherein
the abnormality detection unit generates answer information according to the patient log by using an answer model that has learned an answer of the patient to a question.

5. The information processing device according to claim 4, wherein
the accumulated log reproduction unit presents, in a mode in which a question generated by the doctor and a question based on the observation information are distinguishable, a history of the question in association with the patient log.

6. The information processing device according to claim 4, comprising:
a medical inquiry content accumulation unit that stores a content of the question and an answer of the patient to the question in association with each other.

7. The information processing device according to claim 6, wherein
the medical inquiry content accumulation unit generates a question message regarding a data range designated by the doctor.

8. The information processing device according to claim 7, wherein
the medical inquiry content accumulation unit changes a template of the message on the basis of a designation method of the data range.

9. The information processing device according to claim 1, wherein
the accumulated log reproduction unit presents the abnormality data in a mode that enables distinction from normal data.

10. The information processing device according to claim 1, wherein
the accumulated log reproduction unit presents a tag indicating a content of abnormality in association with the abnormality data.

11. The information processing device according to claim 1, wherein
the accumulated log reproduction unit presents a UI for identifying a normal vital data group from a vital data group of the patient as a normal cluster.

12. The information processing device according to claim 11, wherein
the accumulated log reproduction unit presents a vital data group similar to vital data of a healthy person as a stable candidate, and
the abnormality detection unit identifies the stable candidate approved by a doctor as the normal cluster.

13. The information processing device according to claim 11, wherein
the accumulated log reproduction unit presents, as a stable candidate, a vital data group with a medication period of a medicine designated by a doctor, and
the abnormality detection unit identifies the stable candidate approved by the doctor as the normal cluster.

14. The information processing device according to claim 11, wherein
the accumulated log reproduction unit presents, as a stable candidate, a vital data group similar to a vital data group designated by a doctor, and
the abnormality detection unit identifies the stable candidate approved by the doctor as the normal cluster together with the vital data group designated by the doctor.

15. The information processing device according to claim 11, wherein
the accumulated log reproduction unit presents a data space of the vital data, and the abnormality detection unit identifies a vital data group included in a space designated by a doctor as the normal cluster.

16. An information processing method executed by a computer, the method comprising:
accumulating vital data of a patient for each medication date as a patient log;
detecting abnormality data indicating an abnormal value from the patient log; and
reproducing the patient log so as to have the abnormality data associated with the medication date.

17. A program for causing a computer to implement:
accumulating vital data of a patient for each medication date as a patient log;
detecting abnormality data indicating an abnormal value from the patient log; and
reproducing the patient log so as to have the abnormality data associated with the medication date.
